(19) United States
(12) Patent Application Publication
(10) Pub. No.: US 2026/0157643 A1
(43) Pub. Date: Jun. 11, 2026

(54) BLOOD PRESSURE DETECTION DEVICE AND BLOOD PRESSURE DETECTION METHOD

(71) Applicant: STREAMTECK SCIENTIFIC INC., Taichung City (TW)

(72) Inventor: Yu-Hsiang Su, New Taipei City (TW)

(21) Appl. No.: 19/182,317

(22) Filed: Apr. 17, 2025

Related U.S. Application Data
(63) Continuation-in-part of application No. 18/664,266, filed on May 14, 2024.

(30) Foreign Application Priority Data
May 29, 2024 (TW) 113119899

(51) Int. Cl. A61B 5/021; A61B 5/00; A61B 5/024; A61B 5/352

(52) U.S. Cl. CPC A61B 5/02125; A61B 5/02416; A61B 5/352; A61B 5/7203

(57) ABSTRACT

A blood pressure detection device and a blood pressure detection method are provided. The blood pressure detection method includes: receiving an electrocardiography (ECG) signal and a photoplethysmography (PPG) signal corresponding to the electrocardiography signal; obtaining a feature from the ECG signal and the PPG signal; and inputting the feature into a linear regression model to generate an estimated blood pressure; and outputting the estimated blood pressure.

### CROSS-REFERENCE TO RELATED APPLICATION

This application claims the priority benefit of Taiwan application serial no. 113119899, filed on May 29, 2024. The entirety of the above-mentioned patent application is hereby incorporated by reference herein and made a part of specification.

### BACKGROUND

### Technical Field

The disclosure relates to a physiological parameter measurement technology, and in particular, to a blood pressure detection device and a blood pressure detection method.

### Description of Related Art

Blood pressure is the pressure of blood in the arteries and its unit can be millimeters of mercury (mmHg). Blood pressure is usually expressed in terms of the systolic pressure over diastolic pressure. The systolic pressure is the maximum blood pressure caused by the heart's contraction, and the diastolic pressure is the minimum blood pressure caused by the heart's relaxation.

At present, the blood pressure measuring instrument used in clinical measurement of blood pressure is an electronic blood pressure monitor. When being used to measure the blood pressure, the electronic blood pressure monitor can measure the systolic pressure and the diastolic pressure by constricting or relaxing a cuff worn on the subject's arm. However, the process of constricting the cuff will cause discomfort to the subject, and further, the above method is unable to measure the subject's continuous blood pressure.

Currently, continuous blood pressure measurement can only be performed in an invasive way in medical settings. How to measure the continuous blood pressure of a subject in a non-invasive way is one of the important issues in the field.

### SUMMARY

The disclosure provides a blood pressure detection device and a blood pressure detection method capable of estimating the continuous blood pressure of a subject in a non-invasive manner.

The disclosure provides a blood pressure detection device including a transceiver, a storage medium, and a processor. The storage medium stores a plurality of modules. The processor is coupled to the storage medium and the transceiver and accesses and executes the plurality of modules. The plurality of modules include a communication module, a computing module, and a blood pressure estimator. The communication module receives an electrocardiography (ECG) signal and a photoplethysmography (PPG) signal corresponding to the ECG signal through the transceiver. The computing module obtains a feature from the ECG signal and the PPG signal. The blood pressure estimator inputs the feature into a linear regression model to generate an estimated blood pressure. The communication module outputs the estimated blood pressure through the transceiver.

In an embodiment of the disclosure, the communication module receives a plurality of ECG signals, a plurality of PPG signals corresponding to the plurality of ECG signals, and a plurality of blood pressures corresponding to the plurality of ECG signals through the transceiver. The computing module generates the linear regression model according to the plurality of ECG signals, the plurality of PPG signals, and the plurality of blood pressures.

In an embodiment of the disclosure, the computing module generates the linear regression model based on a least squares method or maximum likelihood estimation.

In an embodiment of the disclosure, the plurality of modules further include a Kalman filter. The communication module receives a measured blood pressure through the transceiver. The Kalman filter, according to the feature, a coefficient of the linear regression model, the estimated blood pressure, and the measured blood pressure, updates the coefficient.

In an embodiment of the disclosure, the computing module calculates noise according to the feature and historical noise. The computing module inputs the feature, the coefficient, the estimated blood pressure, the measured blood pressure, and the noise into the Kalman filter to output the updated coefficient.

In an embodiment of the disclosure, the Kalman filter includes an adaptive Kalman filter.

In an embodiment of the disclosure, the computing module updates the coefficient based on a predetermined period.

In an embodiment of the disclosure, the plurality of modules further include a preprocessing module. The preprocessing module performs preprocessing on the feature before the feature is input into the linear regression model by the blood pressure estimator.

In an embodiment of the disclosure, the preprocessing includes filtering noise.

In an embodiment of the disclosure, the feature includes at least one of the following: a heart rate, a time delay between an R wave peak of the ECG signal and a midpoint between a peak and a valley of the PPG signal, a time delay between the R wave peak of the ECG signal and a peak of a differential signal of the PPG signal, a time delay between the R wave peak of the ECG signal and the peak of the PPG signal, a time delay between the R wave peak of the ECG signal and the valley of the PPG signal, a time duration between a first pulse peak of the PPG signal and a second pulse valley of the PPG signal, a time duration between a pulse peak and a pulse valley of the PPG signal, a time duration between the first pulse peak and a second pulse peak of the PPG signal, a ratio between a maximum amplitude and a minimum amplitude of the PPG signal, the maximum amplitude of the PPG signal; and the minimum amplitude of the PPG signal.

The disclosure further provides a blood pressure detection method, and the method includes the following steps. An electrocardiography (ECG) signal and a photoplethysmography (PPG) signal corresponding to the electrocardiography signal are received. A feature is obtained from the ECG signal and the PPG signal. The feature is input into a linear regression model to generate an estimated blood pressure. The estimated blood pressure is output.

To sum up, in the disclosure, the blood pressure detection device may extract a plurality of features from an ECG signal or a PPG signal and establish a linear regression model for estimating the blood pressure of a subject based on the plurality of features. The blood pressure detection device may also periodically update the linear regression model, so that the linear regression model adapts to the current physiological state of the subject.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a schematic view illustrating a non-contact detection device for an electrocardiography signal according to an embodiment of the disclosure.

FIG. 2 is a schematic diagram illustrating estimation of an ECG signal through wireless signal measurement according to an embodiment of the disclosure.

FIG. 3 is a schematic diagram illustrating training of a wireless signal encoder according to an embodiment of the disclosure.

FIG. 4 is a schematic diagram illustrating training of a preprocessing module according to an embodiment of the disclosure.

FIG. 5 is a schematic diagram illustrating training of a decoder and an ECG signal encoder according to an embodiment of the disclosure.

FIG. 6 is a flow chart illustrating a non-contact detection method for an ECG signal according to an embodiment of the disclosure.

FIG. 7 is a schematic view illustrating a blood pressure detection device according to an embodiment of the disclosure.

FIG. 8 is a schematic graph illustrating an ECG signal and a photoplethysmography (PPG) signal according to an embodiment of the disclosure.

FIG. 9 is a schematic graph illustrating the PPG signal according to an embodiment of the disclosure.

FIG. 10 is a schematic diagram illustrating a Kalman filter according to an embodiment of the disclosure.

FIG. 11 is a flow chart illustrating a blood pressure detection method according to an embodiment of the disclosure.

### DESCRIPTION OF THE EMBODIMENTS

In order to make the disclosure more comprehensible, several embodiments are described below as examples of implementation of the disclosure. Moreover, elements/components/steps with the same reference numerals are used to represent the same or similar parts in the drawings and embodiments.

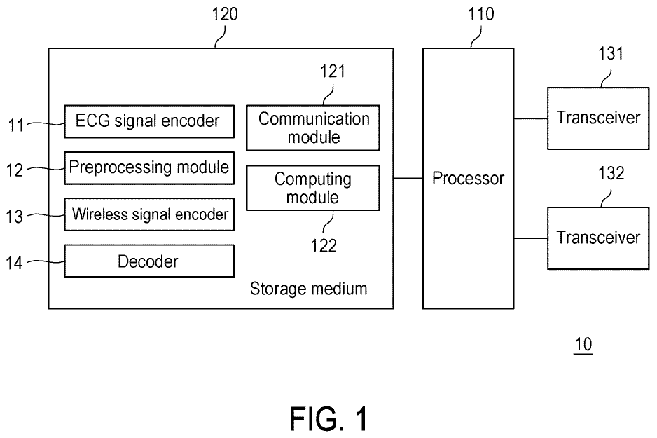

FIG. 1 is a schematic view illustrating a non-contact detection device 10 for an electrocardiography (ECG) signal according to an embodiment of the disclosure. The detection device 10 may include a processor 110, a storage medium 120, a transceiver 131, and a transceiver 132.

The processor 110 may be, for example, a central processing unit (CPU), a programmable micro control unit (MCU) for general or special use, a microprocessor, a digital signal processor (DSP), a programmable controller, an application specific integrated circuit (ASIC), a graphics processing unit (GPU), an image signal processor (ISP), an image processing unit (IPU), an arithmetic logic unit (ALU), a complex programmable logic device (CPLD), a field programmable gate array (FPGA), other similar devices, or a combination of the foregoing devices. The processor 110 may be coupled to the storage medium 120, the transceiver 131, and the transceiver 132 and access and execute a plurality of modules and various application programs stored in the storage medium 120.

The storage medium 120 is, for example, a fixed or movable random access memory (RAM) in any form, a read-only memory (ROM), a flash memory, a hard disk drive (HDD), a solid state drive (SSD), a similar device, or a combination of the foregoing devices and is used to store the plurality of modules or various application programs that can be executed by the processor 110. In this embodiment, the storage medium 120 may store a plurality of modules or models including a communication module 121, a computing module 122, an ECG signal encoder 11, a preprocessing module 12, a wireless signal encoder 13, and a decoder 14, and functions of these modules or models are described in the following paragraphs. The ECG signal encoder 11, the preprocessing module 12, the wireless signal encoder 13, or the decoder 14 may be implemented by a machine learning model such as a deep learning model or a transformer model, but the disclosure is not limited thereto.

The transceiver 131 or the transceiver 132 is used for transmitting and receiving signals. The transceiver 131 or the transceiver 132 may also perform, for example, low noise amplification, impedance matching, frequency mixing, up or down frequency conversion, filtering, amplification, and other similar operations. In an embodiment, the processor 110 may receive a signal from or transmit a signal to an external electronic device through the transceiver 131 or the transceiver 132.

The communication module 121 may transmit a wireless signal to a subject through the transceiver 131 and receive a reflected signal corresponding to the wireless signal. The reflected signal may contain information related to micro-vibrations in the subject's chest. The reflected signal may be used to generate an estimated ECG signal. The signal sent by the transceiver 131 is, for example, a frequency modulated continuous wave (FMCW) signal carried by millimeter waves (mmWave).

The communication module 121 may measure an ECG signal of the subject through the transceiver 132. To be specific, the transceiver 132 may be coupled to one or more electrodes attached to the subject and obtains the ECG signal through the one or more electrodes. The ECG signal may include any one or a combination of 12 sets of lead signals. According to user's needs, each module or model in the detection device 10 may be trained to restore the reflected signal to any one of the 12 sets of lead signals or a combination thereof.

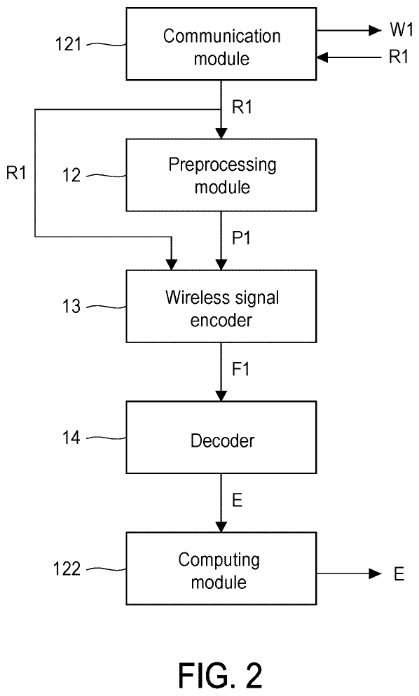

FIG. 2 is a schematic diagram illustrating estimation of an ECG signal E through wireless signal measurement according to an embodiment of the disclosure. The communication module 121 may transmit a wireless signal W1 through the transceiver 131 and receive a reflected signal R1 corresponding to the wireless signal W1. The preprocessing module 12 may perform preprocessing on the reflected signal R1 to generate a processed signal P1. The preprocessing module 12 may capture a signal corresponding to a specific waveform of the ECG from the reflected signal R1 as the processed signal P1. Herein, the specific waveform may include a P wave, a Q wave, an R wave, a S wave, or a T wave.

In an embodiment, before the reflected signal R1 is preprocessed, the preprocessing module 12 may first perform filtering processing on the reflected signal R1 to filter out signals related to the subject's breathing behavior in the reflected signal R1, so that the processed reflected signal R1 only retains information related to the micro-vibrations (i.e., the micro-vibrations associated with the heartbeat) of the subject's chest.

After the processed signal P1 is generated, the wireless signal encoder 13 may perform feature extraction on the processed signal P1 to capture an embedding F1 from the processed signal P1 and the reflected signal R1, where the embedding may also be referred to as a feature vector. Next, the decoder 14 may generate the estimated ECG signal E according to the embedding F1. The computing module 122 may output the estimated ECG signal E for user reference. For instance, the computing module 122 may output the estimated ECG signal E to a display that is communicatively connected to the detection device 10, so as to display the estimated ECG signal E through the display. Based on the above, the detection device 10 may estimate the ECG signal of the subject without the use of a contact sensor.

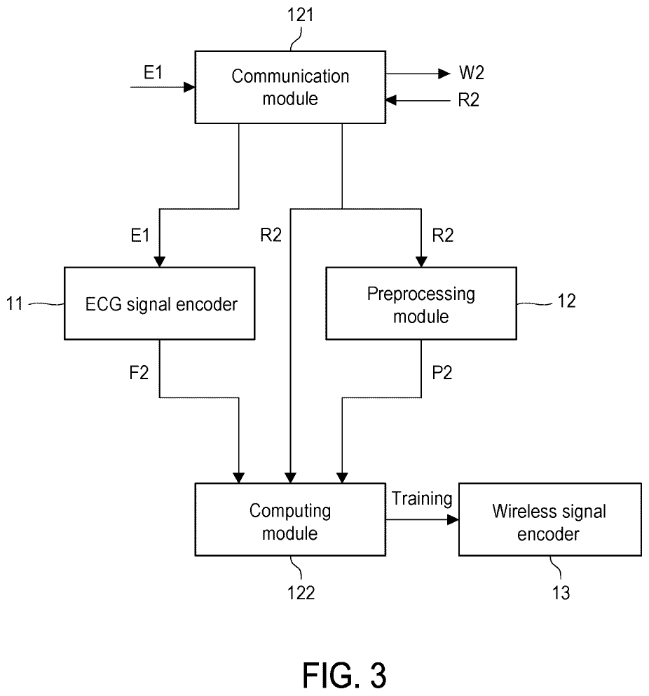

FIG. 3 is a schematic diagram illustrating training of the wireless signal encoder 13 according to an embodiment of the disclosure. The communication module 121 may detect an ECG signal E1 of the subject through the transceiver 132 and the electrodes. The ECG signal E1 is the real ECG signal of the subject. Further, the communication module 121 may transmit a wireless signal W2 to the subject through the transceiver 131 and receive a reflected signal R2 corresponding to the wireless signal W2. That is, the ECG signal E1 and the reflected signal R2 correspond to each other in the time domain.

The ECG signal encoder 11 may perform feature extraction on the ECG signal E1 to capture an embedding F2 from the ECG signal E1. The preprocessing module 12 may perform preprocessing on the reflected signal R2 to generate a processed signal P2. The computing module 122 may train the wireless signal encoder 13 according to the reflected signal R2 and the processed signal P2 based on a machine learning algorithm (e.g., a transformer algorithm). A loss function of the machine learning algorithm may be associated with the embedding F2. The wireless signal encoder 13 may be trained to output an embedding according to the input reflected signal and the processed signal, where the embedding output by the wireless signal encoder 13 is to be similar to the embedding output by the ECG signal encoder 11.

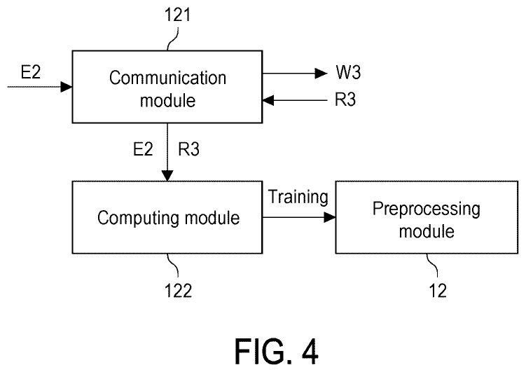

FIG. 4 is a schematic diagram illustrating training of the preprocessing module 12 according to an embodiment of the disclosure. The communication module 121 may detect an ECG signal E2 of the subject through the transceiver 132 and the electrodes. The ECG signal E2 is the real ECG signal of the subject. Further, the communication module 121 may transmit a wireless signal W3 to the subject through the transceiver 131 and receive a reflected signal R3 corresponding to the wireless signal W3. That is, the ECG signal E2 and the reflected signal R3 correspond to each other in the time domain.

The computing module 122 may train the preprocessing module 12 according to the reflected signal R3 based on a machine learning algorithm (e.g., a transformer algorithm). The loss function of the machine learning algorithm may be associated with the ECG signal E2. The pre-processing module 12 may be trained to output a processed signal according to the input reflected signal, and the processed signal output by the pre-processing module 12 is to include a signal corresponding to a specific waveform of the ECG signal (i.e., the P wave, the Q wave, the R wave, the S wave, or the T wave).

To be specific, the computing module 122 may perform peak detection on the ECG signal E2 to detect a time when a specific waveform (i.e., the P wave, the Q wave, the R wave, the S wave, or the T wave) appears in the ECG signal E2. The reflected signal R3 may be used as a data point for label data, and the time associated with the specific waveform in the ECG signal E2 may be used as a label for the label data. The computing module 122 may train the preprocessing module 12 according to the label data. The trained preprocessing module 12 may output the time corresponding to the specific waveform (i.e., the P wave, the Q wave, the R wave, the S wave, or the T wave) according to the input reflected signal.

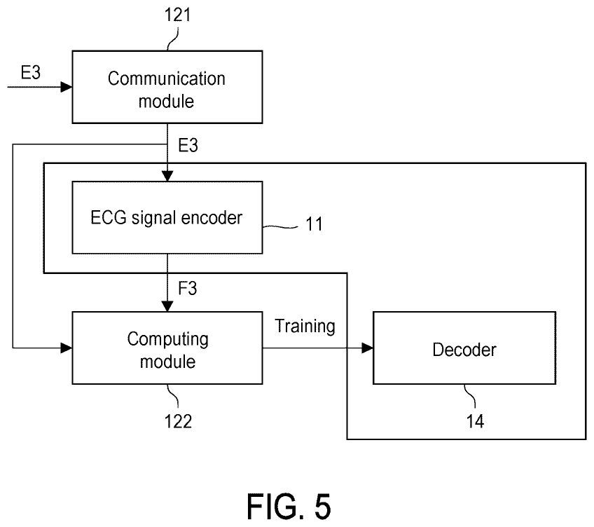

FIG. 5 is a schematic diagram illustrating training of the decoder 14 and the ECG signal encoder 11 according to an embodiment of the disclosure. The communication module 121 may detect an ECG signal E3 of the subject through the transceiver 132 and the electrodes. The ECG signal E3 is the real ECG signal of the subject. The ECG signal encoder 11 may perform feature extraction on the ECG signal E3 to capture an embedding F3 from the ECG signal E3. The computing module 122 may train or update the ECG signal encoder 11 and the decoder 14 according to the ECG signal E3 and the embedding F3 based on a machine learning algorithm (e.g., a transformer algorithm). The loss function of the machine learning algorithm may be associated with the ECG signal E3. The ECG signal encoder 11 and the decoder 14 may be trained, such that the decoder 14 may output an estimated ECG signal based on the embedding output by the ECG signal encoder 11. The estimated ECG signal is to be close to the subject's real ECG signal (e.g., the input of the ECG signal encoder 11).

In an embodiment, the loss function of the machine learning algorithm used to train the decoder 14 may be associated with a spectrum of the ECG signal E3. For instance, the computing module 122 may perform short-time Fourier transform (STFT) on the ECG signal E3 to obtain the spectrum of the ECG signal E3. The loss function may be designed, so that the spectrum (e.g., the spectrum which is obtained by performing STFT on the estimated ECG signal by the computing module 122) of the estimated ECG signal output by the decoder 14 approximates the spectrum of the real ECG signal.

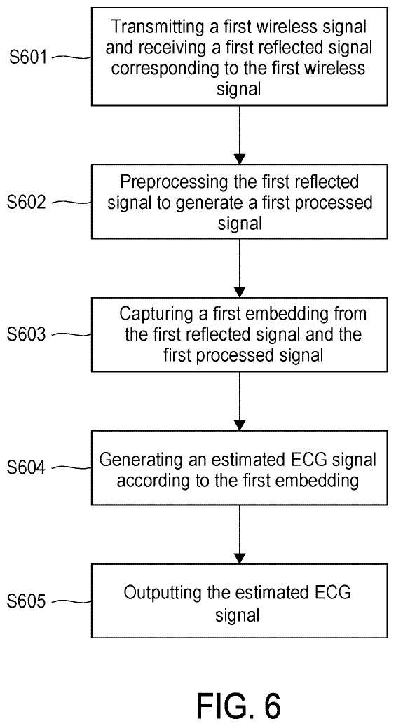

FIG. 6 is a flow chart illustrating a non-contact detection method for an ECG signal according to an embodiment of the disclosure, and the detection method may be implemented by the detection device 10 as shown in FIG. 1. In step S601, a first wireless signal is transmitted, and a first reflected signal corresponding to the first wireless signal is received. In step S602, the first reflected signal is preprocessed to generate a first processed signal. In step S603, a first embedding is captured from the first reflected signal and the first processed signal. In step S604, an estimated ECG signal is generated according to the first embedding. In step S605, the estimated ECG signal is output.

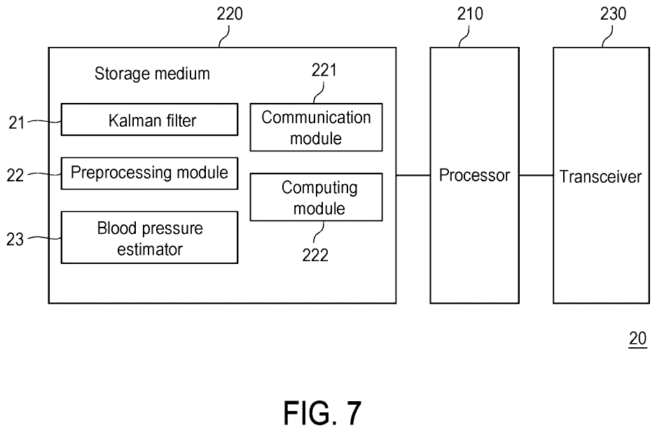

FIG. 7 is a schematic view illustrating a blood pressure detection device 20 according to an embodiment of the disclosure. In an embodiment, the blood pressure detection device 20 may be implemented by the same hardware or software as the detection device 10. In an embodiment, the blood pressure detection device 20 may have all the functions possessed by the detection device 10, and vice versa. The blood pressure detection device 20 may include a processor 210, a storage medium 220, and a transceiver 230. The processor 210 may be coupled to the storage medium 220 and the transceiver 230 and access and execute a plurality of modules and various application programs stored in the storage medium 220. The processor 210, the storage medium 220, and the transceiver 230 may respectively have the same structure or function as the processor 110, the storage medium 120, and the transceiver 131 (or the transceiver 132), so description thereof is not repeated herein. The plurality of modules stored in the storage medium 220 may include a Kalman filter 21, a preprocessing module 22, a blood pressure estimator 23, a communication module 221, and a computing module 222.

The communication module 221 can receive an ECG signal and a photoplethysmography (PPG) signal corresponding to the ECG signal through the transceiver 230. The aforementioned ECG signal is, for example, an estimated ECG signal generated by the detection device 10. The computing module 222 may obtain one or a plurality of features from the ECG signal and the PPG signal. For instance, the computing module 222 may obtain a heart rate (HR) or a pulse transient time (PTT) from the ECG signal and the PPG signal. The pulse transit time may include a time delay of PTTₚ, PTTₘ, PTT_{f}, or PTT_{d} as described in the following paragraphs.

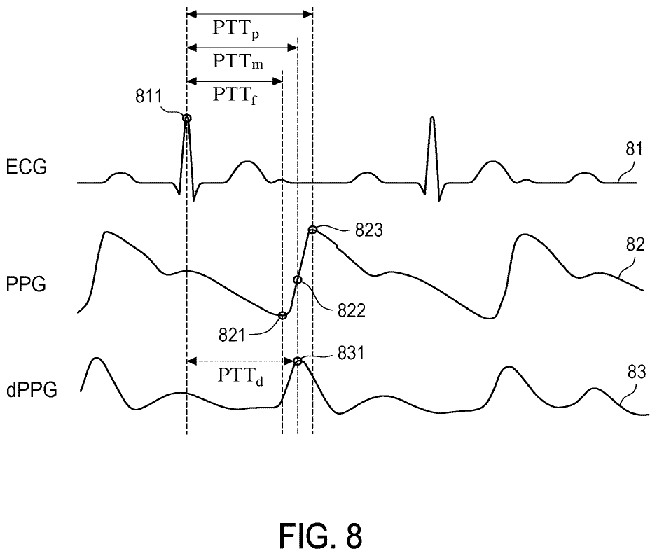

FIG. 8 is a schematic graph illustrating an ECG signal and a PPG signal according to an embodiment of the disclosure. The computing module 222 obtains features from the ECG signal and the PPG signal. In an embodiment, the features may further include a time delay PTTₘ between an R wave peak 811 and a midpoint 822 of an ECG signal 81. Herein, the midpoint 822 is the midpoint between a peak 823 (or a maximum value) and a valley 821 (or a minimum value) of a PPG signal 82 (i.e., a time point of the midpoint 822 is the midpoint between a time point of the valley 821 and a time point of the peak 823).

In an embodiment, the features may further include a time delay PTT_{d} between the R wave peak 811 of the ECG signal 81 and a peak 831 of a differential signal 83 (i.e., the dPPG signal) of the PPG signal 82.

In an embodiment, the features may further include a time delay PTTₚ between the R wave peak 811 of the ECG signal 81 and the peak 823 (or the maximum value) of the PPG signal 82.

In an embodiment, the features may further include a time delay PTT_{f} between the R wave peak 811 of the ECG signal 81 and the valley 821 (or the minimum value) of the PPG signal 82.

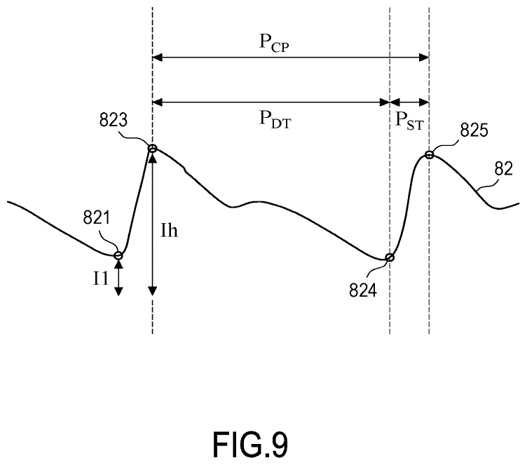

FIG. 9 is a schematic graph illustrating the PPG signal 82 according to an embodiment of the disclosure. In an embodiment, the features may further include peak intensity Ih. The peak intensity Ih is, for example, maximum amplitude of a pulse in the PPG signal 82 (e.g., the value of the peak 823).

In an embodiment, the features may further include valley intensity Il. The valley intensity Il is, for example, minimum amplitude of the pulse in the PPG signal 82 (e.g., the value of the valley 821).

In an embodiment, the features may further include a photoplethysmogram intensity ratio (PIR), where the PIR may be used to represent an arterial diameter change. For instance, PIR is a ratio between the maximum amplitude and the minimum amplitude of the pulse in the PPG signal 82, such as the ratio between the peak 823 and the valley 821.

In an embodiment, the features may further include a cardiac cycle (CP). The cardiac period is, for example, a time duration P_{CP} between a pulse peak (e.g., the peak 823) and an adjacent pulse peak (e.g., a peak 825) of the PPG signal 82.

In an embodiment, the features may further include a diastolic time (DT). The diastolic time is, for example, a time duration P_{DT} between the pulse peak (e.g., the peak 823) and an adjacent pulse valley (e.g., a valley 824) of the PPG signal 82.

In an embodiment, the features may further include a systolic upstroke time (ST). The systolic upstroke time is, for example, a time duration P_{ST} between the pulse valley (e.g., the pulse valley 824) and the pulse peak (e.g., the peak 825) of the PPG signal 82.

After obtaining one or more features, the blood pressure estimator 23 may input the one or more features into a linear regression model to generate an estimated blood pressure. The computing module 222 may output the estimated blood pressure through the transceiver 230 for user reference.

In an embodiment, before the blood pressure estimator 23 inputs one or more features into the linear regression model, the preprocessing module 22 may perform preprocessing on the one or more features. Herein, the preprocessing may include but not limited to filtering noise.

The computing module 222 may be configured to train the linear regression model. To be specific, the communication module 221 may receive a plurality of ECG signals, a plurality of PPG signals corresponding to the plurality of ECG signals, and a plurality of blood pressures corresponding to the plurality of ECG signals through the transceiver 230. The blood pressures are measured by, for example, a conventional electronic blood pressure monitor. The computing module 222 may generate the linear regression model based on the plurality of ECG signals, the plurality of PPG signals, and the plurality of blood pressures based on methods such as a least squares method or maximum likelihood estimation (MLE), as shown in formula (1), where k is a time index, m is a feature index, M is the total number of features, [symbol] is the linear regression model corresponding to the time index k and represents the estimated blood pressure, αₖ=[α_{0,k} α_{1,k} α_{2,k} . . . α_{m,k}] is a coefficient set corresponding to the time index k, and vₖ=[v_{1,k} v_{2,k} . . . v_{m,k}] is a feature set corresponding to the time index k.

$$= {α}_{0 , k} + {\sum }_{m = 1}^{M} {α}_{m , k} {v}_{m , k} \qquad ( 1 )$$

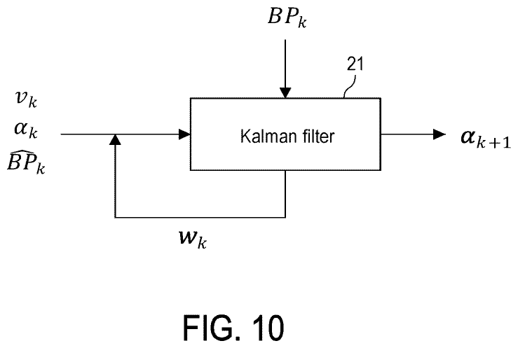

In an embodiment, the computing module 222 may update the coefficient set αₖ of the linear regression model based on a predetermined period, so that the linear regression model adapts to the current physiological state of the subject. FIG. 10 is a schematic diagram illustrating the Kalman filter 21 according to an embodiment of the disclosure. The Kalman filter 21 is, for example, an adaptive Kalman filter.

It is assumed that the current time point corresponds to the time index k. In order to obtain an updated coefficient set αₖ₊₁ corresponding to the time index k+1, the communication module 221 may receive a measured blood pressure BPₖ through the transceiver 230. Herein, the measured blood pressure BPₖ is measured at a time point corresponding to the time index k by, for example, a conventional electronic blood pressure meter. The computing module 222 may calculate noise wₖ corresponding to the time index k according to one or more features in the feature set vₖ and the historical noise (e.g., noise wₖ₋₁ corresponding to the time index (k−1)). In an embodiment, the noise wₖ may be proportional to the heart rate, the pulse transient time, or the historical noise wₖ₋₁.

Next, the computing module 222 may input one or more features in the feature set Ok, the coefficient set αₖ, the estimated blood pressure [symbol] , the measured blood pressure BPₖ, and the noise wₖ into the Kalman filter 21 to update the coefficient set αₖ, and the coefficient set αₖ₊₁ is thereby generated. The computing module 222 may generate a linear regression model [symbol] corresponding to the time index (k+1) according to the coefficient set αₖ₊₁. The linear regression model [symbol] is applied to generate the subject's estimated blood pressure based on the feature set obtained after the time index k.

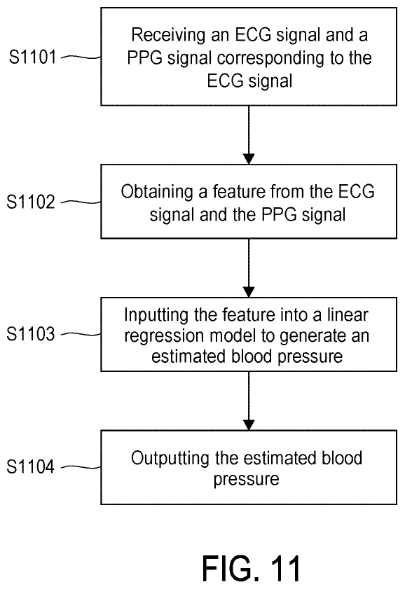

FIG. 11 is a flow chart illustrating a blood pressure detection method according to an embodiment of the disclosure. The blood pressure detection method may be implemented by the blood pressure detection device 20 as shown in FIG. 7. In step S1101, an ECG signal and a PPG signal corresponding to the ECG signal are received. In step S1102, a feature is obtained from the ECG signal and the PPG signal. In step S1103, the feature is input into a linear regression model to generate an estimated blood pressure. In step S1104, the estimated blood pressure is output.

In view of the foregoing, in the disclosure, the blood pressure detection device may extract multiple features from the ECG signal and the PPG signal and establish a linear regression model of blood pressure based on the multiple features. The linear regression model generates the estimated blood pressure based on the measured ECG and PPG signals. Therefore, the blood pressure detection device may obtain the estimated blood pressure of the subject in a non-invasive manner. Further, the subjects' physiology may change over time, so the linear regression model becomes less accurate. To solve the above problem, the blood pressure detection device may update the coefficients of the linear regression model through the Kalman filter, so as to maintain the effectiveness of the linear regression model for the subject.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A blood pressure detection device, comprising:
a transceiver;
a storage medium storing a plurality of modules; and
a processor coupled to the storage medium and the transceiver and accessing and executing the plurality of modules, wherein the plurality of modules comprise:
a communication module receiving an electrocardiography (ECG) signal and a photoplethysmography (PPG) signal corresponding to the electrocardiography signal through the transceiver;
a computing module obtaining a feature from the electrocardiography signal and the photoplethysmography signal; and
a blood pressure estimator inputting the feature into a linear regression model to generate an estimated blood pressure, wherein
the communication module outputs the estimated blood pressure through the transceiver.

2. The blood pressure detection device according to claim 1, wherein
the communication module receives a plurality of electrocardiography signals, a plurality of photoplethysmography signals corresponding to the plurality of electrocardiography signals, and a plurality of blood pressures corresponding to the plurality of electrocardiography signals through the transceiver, wherein
the computing module generates the linear regression model according to the plurality of electrocardiography signals, the plurality of photoplethysmography signals, and the plurality of blood pressures.

3. The blood pressure detection device according to claim 2, wherein
the computing module generates the linear regression model based on a least squares method or maximum likelihood estimation.

4. The blood pressure detection device according to claim 2, wherein the plurality of modules further comprise:
a Kalman filter, wherein
the communication module receives a measured blood pressure through the transceiver, wherein
the Kalman filter, according to the feature, a coefficient of the linear regression model, the estimated blood pressure, and the measured blood pressure, updates the coefficient.

5. The blood pressure detection device according to claim 4, wherein
the computing module calculates noise according to the feature and historical noise, wherein
the computing module inputs the feature, the coefficient, the estimated blood pressure, the measured blood pressure, and the noise into the Kalman filter to output the updated coefficient.

6. The blood pressure detection device according to claim 4, wherein the Kalman filter comprises an adaptive Kalman filter.

7. The blood pressure detection device according to claim 4, wherein
the computing module updates the coefficient based on a predetermined period.

8. The blood pressure detection device according to claim 1, wherein the plurality of modules further comprise:
a preprocessing module performing preprocessing on the feature before the feature is input into the linear regression model by the blood pressure estimator.

9. The blood pressure detection device according to claim 8, wherein the preprocessing comprises filtering noise.

10. The blood pressure detection device according to claim 1, wherein the feature comprises at least one of the following: a heart rate; a time delay between an R wave peak of the electrocardiogram signal and a midpoint between a peak and a valley of the photoplethysmography signal; a time delay between the R wave peak of the electrocardiogram signal and a peak of a differential signal of the photoplethysmography signal; a time delay between the R wave peak of the electrocardiogram signal and the peak of the photoplethysmography signal; a time delay between the R wave peak of the electrocardiogram signal and the valley of the photoplethysmography signal; a time duration between a first pulse peak of the photoplethysmography signal and a second pulse valley of the photoplethysmography signal; a time duration between a pulse peak and a pulse valley of the photoplethysmography signal; a time duration between the first pulse peak and a second pulse peak of the photoplethysmography signal; a ratio between a maximum amplitude and a minimum amplitude of the photoplethysmography signal; the maximum amplitude of the photoplethysmography signal; and the minimum amplitude of the photoplethysmography signal.

11. A blood pressure detection method, comprising:
receiving an electrocardiography (ECG) signal and a photoplethysmography (PPG) signal corresponding to the electrocardiography signal;
obtaining a feature from the electrocardiography signal and the photoplethysmography signal;
inputting the feature into a linear regression model to generate an estimated blood pressure; and
outputting the estimated blood pressure.
